(19) United States
(12) Patent Application Publication
(10) Pub. No.: US 2019/0365934 A1
(43) Pub. Date: Dec. 5, 2019

(54) METHODS FOR IN VIVO MONITORING OF DOPAMINERGIC DISORDERS AND EFFICACY OF TREATMENT AGENTS THEREFOR

(71) Applicant: LIKEMINDS, INC., Auburndale, MA (US)

(72) Inventor: KENNETH L. RICE, Auburndale, MA (US)

(73) Assignee: Kenneth L. Rice, Auburndale, MA (US)

(21) Appl. No.: 16/485,228

(22) PCT Filed: Feb. 9, 2018
(86) PCT No.: PCT/US18/17571

§ 371 Date: Aug. 12, 2019

Related U.S. Application Data
(60) Provisional application No. 62/457,328, filed on Feb. 10, 2017.
(60) Provisional application No. 62/465,910, filed on Mar. 2, 2017.

(51) Int. Cl. A61K 51/04

(52) U.S. Cl. CPC A61K 51/0448

(57) ABSTRACT

Provided are methods of evaluating an ability of a therapeutic composition to modulate a dopaminergic activity in an organ of a mammalian subject having a dopaminergic disorder. Also provided are methods of assessing and monitoring the progression of a dopaminergic disorder in tan organ of a mammalian subject, and of optimizing the treatment of such a disorder.

### CROSS-REFERENCE TO RELATED APPLICATIONS

This application claims priority to and the benefit of Provisional Application Nos. 62/457,328 filed Feb. 10, 2017 and 62/465,910, filed Mar. 2, 2017. The entire teachings of the above applications are incorporated herein by reference.

### FIELD OF THE INVENTION

The present disclosure relates to human and veterinary medicine. More specifically, the disclosure relates to the diagnosis, and to the monitoring of treatment of dopaminergic disorders by imaging neuron dopamine transporters.

### BACKGROUND

The dopamine transporter or “DaT” (also dopamine active transporter, SLC6A3) is a membrane-spanning protein that pumps the dopamine out of the synaptic cleft and back into the cytosol. In the cytosol, other transporters sequester the dopamine into cellular vesicles for storage and later release. Dopamine reuptake via DaT provides the primary mechanism through which dopamine is cleared from the synapse.

DaT molecules are found in many organs of the mammalian body, e.g., brain, pancreas, kidney small intestine, thyroid, ovary, and lung. DaT has also been implicated in a number of disorders affecting these organs (e.g. Parkinson's disease (PD), obsessive compulsive disorder (OCD), post-traumatic stress disorder (PTSD), renal cell carcinoma, osmotic imbalance, Hartnup disease, etc.).

Neurological disorders, and in particular, dopaminergic disorders, often display a variety of clinical symptoms. For example, PD patient would typically exhibit bradykinesia, tremor, rigidity, and potentially postural instability. PD tends to be diagnosed later in life, such as after age 60, and its clinical symptoms tend to get progressively worse, ending in partial or total incapacitation of the patient.

One way to clinically assess the symptoms in the case of PD in vivo is to rely on a version of the unified PD rating scale (UPDRS), which allows a clinician to obtain a score for the clinical severity of the disease by observing a set of motor functions. Even though it is commonly used, this scale is not without drawbacks.

The underlying mechanisms of the clinical progression of PD are not well understood. It appears that the clinical stage of the disease correlates to the loss of dopaminergic neurons in a region of the brain called Substantia Nigra Pars Compacta (SNc). SNc is one of the two components of substantial nigra (SN), which itself is a part of the basal ganglia, where it normally is in communication with the striatum (especially the dorsal striatum), which is also a part of the basal ganglia. The death of dopaminergic neurons from the area of the SNc results in a decrease in the number of dopamine transporter (DaT) molecules, resulting in a decrease in dopamine supply available for healthy neuronal function. Therefore, assessing the level of DaT is one potential approach to study the disease stage.

A complication with many neurological disorders, as exemplified by PD, is that the disease onset in vivo occurs usually many years before the patient displays symptoms of the disease. For example, in PD, the period between disease onset through mechanisms in the brain and a diagnosis through clinical symptoms can be as much as 20 years. Estimates indicate that typical disease symptoms appear when 50% of SN cells have been lost and when merely 20% of striatal dopamine neurotransmitter remains. For that reason, it is often too late for any therapeutic regimen to be effective once a clinical diagnosis based on disease symptoms is made.

The most common attempted treatment for PD is dopamine replacement therapy (DRT), which may involve administration of one of the following four types of neuroprotective agents: (1) a dopamine precursor such as levodopa (L-DOPA); (2) a dopamine agonist (DA); (3) a monoamine oxidase B inhibitor; and (4) a catechol-O-methyltransferase inhibitor. To a limited degree, DRT is able to slow down the degeneration in the striatal plasticity that accompanies PD. For each treatment, an earlier diagnosis is more likely to improve the results. Alternative treatment options include those that target oxidative stress, protein mis-folding and aggregation (especially of alpha-synuclein), neuroinflammation, and apoptosis. Further options may include surgical methods, neural transplantation, stem cell therapy, and gene therapy.

Unfortunately, current approaches for diagnosing and treating PD can result in numerous problems, as many of its symptoms are shared between multiple neurological disorders. Among the conditions that are often confused with each other are PD, essential tremor, vascular Parkinsonism, drug-induced Parkinsonism, corticobasal degeneration (CBD), progressive supranuclear palsy (PSP), multiple system atrophy (MSA), and Lewy body dementia (LBD). Accordingly, the rate of misdiagnosis in vivo is quite high.

This difficulty in obtaining an accurate diagnosis solely based on clinical symptoms results in heterogeneous patient populations with different underlying disorders having the common clinical symptom of tremor. For example, a set of PD patients so diagnosed based on motor functions likely would include some members that have LBD. Not only is an accurate diagnosis of the underlying pathology leading to the clinical symptom of tremor difficult to obtain and fraught with misdiagnoses, the late diagnosis makes it often impossible to effectively treat a patient and influence the progression of the underlying disease.

These problems lead to additional ones. For example, due to a late diagnosis of any disorder, information related to the full time course of the pathological progress of the disease is typically missing. In addition, due to the subjective nature of assessing motor functions, measured or determined either by physical observation or patient interview, the diagnostic results can be inconsistent between different assessments and different diagnosing health professionals. Furthermore, due to the heterogeneity in the identified patient population exhibiting clinical signs of tremor, the very process of finding a cure is compromised, since the efficacy and effectiveness studies cannot be reliably performed on an incorrectly identified patient population. Moreover, the currently available most advanced diagnostic methods, for example, neuro-imaging, are time-consuming, which not only decrease the frequency with which clinicians rely on them, but which may also increase their cost to patients.

In addition, an inaccurate diagnosis may result in a treatment which is ineffective or even harmful. Also, even with an accurate diagnosis, the determination of the effectiveness of a particular therapeutic drug or drug dosage is difficult to determine.

Thus, more accurate methods of diagnosing a dopaminergic disorder and its progression, and companion methods of determining if the treatment provided has been officious, are needed.

### SUMMARY

It has been discovered that an imaging method that quantifies the level of DaT in neurons in vivo can be used to accurately and positively diagnose in vivo (in some cases, in less than an hour of imaging time), if a patient has a malfunction of dopaminergic neurons resulting in the clinical occurrence of a dopaminergic disorder such as PD. It has further been discovered that using a series of repeated scans in vivo enables observation of disease progression over time by reliance on an objective criterion, i.e., imaging of activity of dopaminergic neurons in the brain of the patient in vivo. Because DaT is found in the brain and in a number of organs in the body, this method allows accurate longitudinal documentation of dopaminergic-related disease progression on a cellular level and the efficacy of pharmacologic or non-pharmacologic interventions designed to modify disease progression.

These discoveries have been exploited to provide the present disclosure, which, in part, provides an in vivo assay method for evaluating a therapeutic composition for its ability to modulate a dopaminergic activity in an organ of a mammalian subject having a dopaminergic disorder. The method comprises administering radiolabeled tropane to the subject; determining a baseline level and pattern of binding of the administered radiolabeled tropane to dopamine transporters (DaT) in a the organ of the subject; treating the subject with an initial dose of a first therapeutic composition; administering a radiolabeled tropane to the treated subject; and determining the level and/or pattern of radiolabeled tropane binding to the organ of the treated subject. A change in level and/or pattern of radiolabeled tropane binding in the treated subject relative to baseline levels and/or patterns of radiolabeled tropane binding is indicative of the ability of the therapeutic composition to modulate a dopaminergic activity in the organ of the subject.

In some embodiments, the change in the level of tropane binding is a decrease or increase of at least about 5% to at least about 10%, at least about 5%, at least about 6%, at least about 7%, at least about 8%, at least about 9%, or at least about 10%

In certain embodiments, the organ affected by the dopaminergic disorder is the brain. In other embodiments, the affected organ is the lung, kidney, pancreas, testes, ovary, or thyroid.

In some embodiments, the tropane administered is 2-carbomethoxy-3-(4-fluorophenyl)-N-(1-haloprop-1-en-3-yl)nortropane (DaT2020), 2-carbomethoxy-3-(4-fluorophenyl)-N-(1-iodoprop-1-en-3-yl)nortropane, or [I-123] N-ω-fluoropropyl-2β-carbomethoxy-3β-(4-iodophenyl) nortropane (DaTscan).

In some embodiments, the dopaminergic disorder is Parkinson's disease, attention deficit hyperactivity disorder, dementia, clinical depression, schizophrenia, or addictive disorder (drugs, smoking). In other embodiments, the dopaminergic disorder is Hartnup disease, diabetes, type 1, renal cell carcinoma, or small lung cell carcinoma, and other cancers containing DaT transporters for their physiologic activity as cancer cells.

In particular embodiments, the tropane is radiolabeled with ¹²³I, ¹²⁴I, ¹²⁵I, ¹⁸F, ^{99m}Tc, ¹¹C, or ^{117m}Sn. In certain embodiments, the tropane is radiolabeled with ¹²³I, ¹²⁴I, ¹²⁵I, or ^{99m}Tc, or ^{117m}Sn, and the level and pattern of binding of radiolabeled tropane is measured by SPECT. In other embodiments, the tropane is radiolabeled with ¹⁸F, ¹²⁴I, and ¹¹C, and the level and pattern of binding of radiolabeled tropane is measured by PET.

In certain embodiments, the method further comprises the steps of: treating the subject with a secondary dose of the therapeutic composition which is different from the initial treatment dose if the level of radiolabeled tropane binding is decreased; administering the radiolabeled tropane to the subject treated with the secondary dose; and measuring the level of radiolabeled tropane binding. An increase or decrease in the level of tropane binding of at least about 5% to at least about 10%, 5%, at least about 6%, at least about 7%, at least about 8%, at least about 9%, or at least about 10% in the level of radiolabeled tropane binding is indicative of the ability of the secondary dose of the therapeutic composition to modulate dopaminergic activity in the organ.

In some embodiments, the method further comprises; treating the subject with a second therapeutic composition which is different from the first therapeutic composition; administering radiolabeled tropane to the subject treated with the second therapeutic composition; and measuring the level and/or pattern of radiolabeled tropane binding. A change of at least about 5% to at least about 10%, 5%, at least about 6%, at least about 7%, at least about 8%, at least about 9%, or at least at least 10% in the level and/or pattern of radiolabeled tropane binding being indicative of the ability of the first and second therapeutic compositions to modulate dopaminergic activity in the organ.

In other embodiments, the treating step comprises treating the subject with the first therapeutic composition and with a second therapeutic composition which is different from the first therapeutic composition. A change in the level and/or pattern of radiolabeled tropane binding of at least about 5% to at least about 10%, at least about 5%, at least about 6%, at least about 7%, at least about 8%, at least about 9%, or at least at least about 10% is indicative of the ability of the first and second therapeutic compositions to modulate dopaminergic activity in the organ.

The disclosure also provides an in vivo method of obtaining a time course for the progression of a dopaminergic disorder in an organ of a mammalian subject. The method comprises: carrying out a detection process, the detection process comprising; administering a radiolabeled tropane to the subject; and acquiring a first tomographic image of the organ. The detection process is then repeated to obtain a second tomographic image of the organ. A difference in the second tomographic image relative to the first tomographic image is indicative of a change in the progression of the dopaminergic disorder in the organ of the subject.

In some embodiments, the difference in the second tomographic image relative to the first is about at least about 5% to at least about 10%, at least about 5%, at least about 6%, at least about 7%, at least about 8%, at least about 9%, or at least about 10%.

In certain embodiments, the detection process is performed twice after acquiring the first and second tomographic images, to obtain a third tomographic image, A difference in the third tomographic image relative to the first and/or second tomographic image is indicative of a change in the progression of the dopaminergic disorder in the organ of the subject. In other embodiments, the detection process is repeated multiple times to acquire multiple tomographic images, a difference in at least one of the multiple tomographic images relative to the first, second, and/or third tomographic image being indicative of a change in the progression of the dopaminergic disorder in the organ of the subject.

In some embodiments, the tropane is 2-carbomethoxy-3-(4-fluorophenyl)-N-(1-haloprop-1-en-3-yl) nortropane, tropane is 2-carbomethoxy-3-(4-fluorophenyl)-N-(1-iodoprop-1-en-3-yl) nortropane, or N-ω-fluoropropyl-2β-carbomethoxy-3β-(4-iodophenyl) nortropane (DaTscan).

In certain embodiments, the radiolabel of the tropane is ¹²³I, and in some embodiments the acquiring step is performed via SPECT. In other embodiments, the radiolabel is ¹²⁴I or ^{117m}Sn, and in certain embodiments, the acquiring step is performed via PET.

In another aspect, the present disclosure provides an in vivo method of optimizing a treatment regimen of a therapeutic formulation for a dopaminergic disorder in an organ of a mammalian subject. The method comprises administering a radiolabeled tropane to the subject treated with a first dose of the therapeutic formulation; measuring a level of tropane binding to the organ by acquiring a tomographic image; comparing the level of tropane binding to a projected level of tropane binding obtained from at least one tomographic image acquired before the administering step, and administering to the subject a second dose of the therapeutic formulation different than the first dose if the level of tropane is different than a projected level of tropane binding.

In some embodiments, the level of tropane is at least about 5% to a least about 10%, at least about 5%, at least about 6%, at least about 7%, at least about 8%, at least about 9%, or 10% different than a projected level of tropane binding

In particular embodiments, the tropane is 2-carbomethoxy-3-(4-fluorophenyl)-N-(1-iodoprop-1-en-3-yl) nortropane (DaT2020) or 2-carbomethoxy-3-(4-fluorophenyl)-N-(1-iodoprop-1-en-3-yl) nortropane.

In certain, embodiments, the radiolabel of the tropane comprises ¹²³I, ¹²⁴, or ^{117m}Sn, and in some embodiments, the tomography is SPECT. In other embodiments, the tropane is [I¹²³]-N-ω-fluoropropyl-2β-carbomethoxy-3β-(4-iodophenyl) nortropane (DaTscan), and the tomography is SPECT.

The disclosure also provides an in vivo method of determining effectiveness of a neuroprotective agent in a treatment of a mammalian subject having a dopaminergic disorder in an organ. The method comprises: administering to the subject treated with the neuroprotective agent a radiolabeled tropane; acquiring a tomographic image of the organ via SPECT or PET; measuring a level of tropane binding in the organ from the tomographic image; and determining the effectiveness of the neuroprotective agent based on the level of tropane binding in the organ relative to a projected level of tropane binding, the projected level of binding being obtained from at least one previously acquired obtained tomographic image. A difference in the level of tropane binding of at least about 5% to at least about 0%, at least about 5%, at least about 6\hat{ }, at least about 7%, at least about 8%, at least about 9%, or at least about 10% is indicative of the effectiveness of the neuroprotective agent.

In some embodiments, the difference in the level of tropane binding is an increase in tropane binding or is a decrease in tropane binding.

In certain embodiments, the projected level is obtained from two or more previously acquired tomographic images, and wherein the projected level is obtained through a regression analysis of levels found in the previously obtained tomographic images.

In some embodiments, the tropane is 2-carbomethoxy-3-(4-fluorophenyl)-N-(1-iodoprop-1-en-3-yl) nortropane (Dat2020) or 2-carbomethoxy-3-(4-fluorophenyl)-N-(1-iodoprop-1-en-3-yl) nortropane.

In some embodiments, the radiolabel of the tropane comprises ¹²³I, ¹²⁴I, or ^{117m}Sn. In particular embodiments, the tropane is I^{123]}-N-ω-fluoropropyl-2β-carbomethoxy-3β-(4-iodophenyl) nortropane (DaTscan), and the tomography is SPECT.

In yet another aspect, the disclosure provides an in vivo method of detecting binding of a radiolabeled tropane to dopamine transporter (DaT) molecules in the brain of a mammalian subject. This method comprises administering the radiolabeled tropane to a subject; initiating the acquisition of a tomographic image about 15 minutes after administering the tropane; and terminating the tomographic image acquisition about 5 minutes to about 10 minutes after initiation, a pattern of tropane binding to DaT molecules in the brain being obtained having two comma-shaped regions that are bilaterally symmetric with each if the brain of the subject is not affected by a dopaminergic disorder of the brain. If the brain is affected by a dopaminergic disorder, the pattern is different and may be asymmetrical.

In some embodiments, the tropane is 2-carbomethoxy-3-(4-fluorophenyl)-N-(1-iodoprop-1-en-3-yl) nortropane or 2-carbomethoxy-3-(4-fluorophenyl)-N-(1-iodoprop-1-en-3-yl) nortropane, or N-ω-fluoropropyl-2β-carbomethoxy-3β-(4-iodophenyl) nortropane. In certain embodiments, the radiolabel of the tropane is ¹²³I, ¹²⁴I, or ^{117m}Sn. In particular embodiments, the acquiring step is performed via SPECT or PET.

The aspects and embodiments described above have various advantages. For example, they allow a clinician to obtain results in a quicker way as compared to the previously available methods. This is partially due to the increased sensitivity of the used tropane and to the tropane's faster passage from the blood-brain barrier. As a result, the methods are also more accurate than the previously available methods.

In addition, the disclosed methods avoid the problems caused by heterogeneous patient populations, concomitantly benefiting drug discovery efforts as well as accurate tuning of treatment regimens by providing an enhanced time course for disease progression. Because the methods can be employed even when the motor symptoms are not detectable, they also allow an earlier diagnosis of a disorder. Furthermore, due to the information-richness of the images, which provide not only a density or amount metric for DaT but also pattern information for DaT, the methods allow a clinician to more accurately distinguish between different disorders.

### DESCRIPTION

The disclosures of any patents, patent applications, and publications referred to herein are hereby incorporated by reference in their entireties into this application in order to more fully describe the state of the art as known to those skilled therein as of the date of the invention described and claimed herein. The instant disclosure will govern in the instance that there is any inconsistency between the patents, patent applications, and publications and this disclosure.

Unless defined otherwise, all technical and scientific terms used herein have the same meaning as commonly understood by one of ordinary skill in the art to which this disclosure belongs. The initial definition provided for a group or term herein applies to that group or term throughout the present specification individually or as part of another group, unless otherwise indicated.

The present invention provides, at least in part, methods of in vivo monitoring dopaminergic disease progression, and of determining the effectiveness of a treatment or the ability of a therapeutic agent to modulate dopaminergic activity in an organ of a mammalian subject using a radiolabeled tropane which binds to DaT Depending on the radiolabel of the tropane, the assay methods can employ SPECT, or PET. The methods also provide for an abbreviated alternative procedure, in which a diagnosis can be attained within 10 minutes after the initial period of about 15 minutes. In addition, the methods provide for monitoring of neuroprotective agent effectiveness though repeated scans.

### 1. Radiolabeled Tropanes

The methods of the present disclosure include the use of the compound 2-carbomethoxy-3-(4-fluorophenyl)-N-(1-haloprop-1-en-3-yl) nortropane (DaT2020), and derivatives thereof, that are radiolabeled with an isotope readable by SPECT or PET. In one specific example, the iodine is ¹²³I, a radioactive isotope. DaT2020 is essentially a tropane that allows one to obtain information about the DaT molecules in the human brain. For example, when the halogen is iodine, DaT2020 specifically corresponds to 2-carbomethoxy-3-(4-fluorophenyl)-N-(1-iodoprop-1-en-3-yl) nortropane. Various tropanes, some of which can at least in part, be deployed for the present invention, are disclosed in U.S. Pat. Nos. 5,493,026; 8,084,018; 8,574,545; 8,986,653, and PCT International Application No. PCT/US2015/037340. A tropane such as [¹²³I]-2β-carbomethoxy-3β-(4-flurophenyl)-N-(3-iodo-E-allyl) nortropane or its various derivatives can therefore be used as well.

For imaging, radiolabeled DaT2020 can be used. Radiolabeled DaT2020 can be commercially obtained (e.g., from Alseres Pharmaceuticals, Auburndale, Mass.) or synthesized (see, e.g., U.S. Pat. Nos. 8,986,653 and 8,574,545). Alternatively, radiolabeled DaT2020 may be generated by the user through a radiolabeling procedure. For example, one may allow a reaction between a haloallyl Sn precursor (pre-DaT2020) and a radionuclide under oxidative conditions. Other standard methods of radiolabeling can be used as well. DaT2020 can be in lyophilized form or in aqueous solution form, but for radiolabeling, pre-DaT2020 in lyophilized form is useful.

Non-limiting examples of some useful SPECT-readable radiotropanes for DaT detection include [¹²³I]-2β-carbomethoxy-3β-(4-iodophenyl)tropane ([¹²³I]-beta-CIT); [¹²³I]-2β-carbomethoxy-3β-(4-iodophenyl)-N-(3-fluoropropyl) nortropane ([¹²³I]-FP-CIT); [¹²³I]-altropane; and [^{99m}Tc]-TRODAT-1. Among these, [¹²³I]-FP-CIT is stable for 4 hours post-injection, has a half-life of about 13 hours, emits gamma rays with energy of 159 keV, and is FDA approved. It can be administered at a dosage of 111 MBq-185 (185) MBq, and a scan dosage of 2.3 mSv to 4.4 mSv.

PET-readable radiotropanes for DaT detection include [¹¹C]2-carbomethoxy-3-(4-18F-fluorophenyl)tropane ([¹¹C]CFT), [¹⁸F]CFT, ¹¹C-2β-Carbomethoxy-3β-(4-tolyl)tropane (¹¹C-RTI-32), [¹⁸F]-FP-CIT, and ¹¹C-methylphenidate. PET can also be used to detect aromatic amino acid decarboxylase (AADC) by using ¹⁸F-3,4-dihydroxyphenylalanine (¹⁸F-DOPA), or vesicle monoamine transporter (VMAT2) by using [¹¹C]dihydrotetrabenazine or [¹⁸F]dihydrotetrabenazine.

Isotopes can be obtained from commercially available sources.

The location of the radioisotope as well as the identity of the radioisotope on DaT2020 can be varied. The isotope can be located at any position on DaT2020 and can be directly linked or indirectly linked via a linker (see, U.S. Pat. No. 8,574,545). One suitable position is the free terminus of the haloallyl moiety.

The methods of the disclosure also include the use of the compound loflupane (FPCIT); [I-123] N-ω-fluoropropyl-2β-carbomethoxy-3β-(4-iodophenyl) nortropane (DaTScan) for SPECT imaging. This compound can be synthesized or commercially obtained from GE Healthcare, Chicago, Ill.

### 2. Methods of Administering Tropane

In order to assess DaT levels in an organ of the body of a mammalian subject, including the brain, other parts of the central nervous system (CNS), and other organs including, but not limited to, kidney, pancreas, lung, testes, ovary, and other cancers that involve dopaminergic receptors and transporters in which the subject is administered a radiolabeled tropane.

Administration of the tropane is via a syringe into the bloodstream of a subject (I.V.), after which it passes into an organ or through the blood-brain barrier and quickly binds to the DaT molecules in the basal ganglia of the subject. Other methods of administration can also be utilized, for example, the direct injection of suitable amounts into brain arteries or into other organ arteries via catheters following established procedures, e.g, in invasive neuroradiology or other known methods.

The dose of tropane to be administered ranges from about 1 mCi to about 10 mCi, from about 5 mCi to about 8 mCi, or about 8 mCi. For example, about 2 mCi to about 6 mCi of DaT2020 or about 5 mCi of DaT2020 can be administered. Alternatively, for example, about 8 mCi of DaTScan can be administered.

In some cases, preliminary steps are performed before the administration of the tropane. For example, for SPECT imaging of DaT binding, steps that counter serotonin-reuptake inhibitors, amphetamines, and sympathomimetics can be employed. In particular, if the isotope is an Iodine isotope, prevention of thyroid uptake of the isotope may be warranted, since some free isotope might exist in the tropane solution. This can be accomplished by orally administering to the subject a Lugol solution, potassium iodide solution, or potassium perchlorate solution. A physician would also be likely to require discontinuance of any medications that might interfere with the binding of the tropane to DaT molecules.

### 3. Methods of Detecting Bound Tropane

Once the subject has been administered the tropane after being positioned on the imaging table, tropane location, and pattern of tropane binding in the body is then determined to assess DaT levels, patterns, and availability. Various methods are available, including magnetic resonance imaging (“MRI”) in its various forms (e.g., cMRI, MRS, DWI/DTI, fMRI); transcranial sonography (TCS), PET, and SPECT. Some useful methods of the present disclosure make use of SPECT, while others make use of PET. Each of these two methods is used with a radiolabeled DaT2020.

SPECT is used to image DaT2020 or DaTScan that is radiolabeled with an isotope that emits gamma radiation. One non-limiting suitable isotope is ¹²³I. Examples of other isotopes that may be used include ¹²⁵I, ^{99m}Tc, ^{117m}Sn, and others. Image acquisition can be started after about 15 minutes. In some cases, a full body SPECT scanner is used, which would typically have a detector diameter that would accommodate a human body. Although a direct interpretation of the images by a clinician may be all that is needed to establish a diagnosis, in some cases, software-based reconstruction algorithms, and filtering methods can be used as well.

PET is used to image DaT2020 that is radiolabeled with an isotope that emits positrons. PET cameras are commercially available (e.g., GE Healthcare, Inc., Chicago, Ill.). Isotopes that can be used for PET include ¹⁸F, ¹²⁴I, and ¹¹C.

Some isotopes emit both gamma rays and positrons (e.g., ¹²⁴I), and thus such isotopes can be suitable for use with both SPECT and PET. In general, PET tends to have a better resolution than SPECT. However, some SPECT methodologies can reach similar resolution levels as PET. From the PET or SPECT data and images, the density of DaT can be quantified by relying on the bound tropanes. Tomographs can be obtained by SPECT and PET and can be produced based on the mathematical procedure tomograph reconstruction. SPECT or PET-computed tomography is produced from multiple projectional images, and many known reconstruction algorithms can be used.

One exemplary way to quantify DaT density is through computing binding potential. Binding potential is the maximum number of binding sites (Bₘₐₓ) divided by dissociation constant (K_{d}). The binding potential can be calculated from a continuous scan starting at about 15 minutes post tropane administration. A region of interest can be identified and the counts in that region can be determined. Numerical values for binding potential can be calculated using appropriate modeling and these values can be compared among treatments and along a time course. The striatal binding potential of ¹²³I-Altropane (k3/k4) can be calculated by the reference region approach as described by Farde, et al. (J. Cereb. Blood Flow Metab. (1989) 9:696-708).

### 4. Methods of Determining a Baseline Level and Pattern of Tropane Binding

The disclosed methods provide a method of detecting binding of a radiolabeled tropane to dopamine transporter (DaT) molecules in the brain of a mammalian subject, which can be used to determine if a mammalian subject has a dopaminergic disorder in an organ and to provide a baseline level of binding.

For example, when the organ is the brain, a normal pattern of tropane binding typically consisting two comma-shaped regions that are bilaterally symmetric is obtained by administering the radiolabeled tropane to a subject. The clinician initiates tomatography image acquisition about 15 minutes after administering the radiolabeled tropane to the subject, and terminates acquisition about 5 minutes to about 10 minutes after initiation in an abbreviated procedure (or within about 30 minutes for the full procedure). If the images do not clearly seem to be comma shaped (e.g., they are fuzzy or are somewhat round shaped), the clinician may choose to continue the image acquisition for up to about 30 total minutes after the initial 15 minute period. If the brain is affected by a dopaminergic disorder, the pattern of binding obtained may be asymmetrical not having a comma shape. The method provides a baseline level of tropane binding.

In another assay, the clinician starts image collection about 180 minutes after administering I.V. DaT scan to a subject. Thereafter, the image acquisition can be completed when at least 1.5×10⁶ w counts are collected (e.g., within about 45 minutes).

A baseline level, depending on the application of the method, may correspond to the level (e.g., as an averaged density, as a summed total, or as a two-dimensional distribution) of DaT binding in a subject that is free of the neurological disorder of concern, or of DaT binding in a subject before the administration of a potential neuroprotective agent. Apart from the character of the subject or the stage of imaging of the subject, patterns of binding and levels of binding can be determined similarly for baseline determinations as well as for effectiveness determinations.

With respect to binding patterns, in the brain a symmetric, comma-shaped pattern of DaT binding often indicates that the dopaminergic functioning in the brain is normal. Thus, a comma-shaped pattern would typically rule out Parkinsonian disorders (e.g., PD, MSA, and PSP) and LBD. However, even though a subject with a comma-shaped pattern may be healthy, he may also have a different non-Parkinsonian condition (e.g., essential tremor, Alzheimer's disease). In contrast, in a subject with a Parkinsonian syndrome, the DaT binding pattern is often asymmetric, period shaped (e.g., one side somewhat circular, while the other side is somewhat triangular).

In other organs unaffected by a dopaminergic disorder and where DaT binding is measurable, such as in kidney, lung, pancreas, testes, and ovary, certain patterns of binding are obtained and used to compare with patterns obtained from organs affected by a dopaminergic disorder.

### 5. Monitoring of Clinical Disease Progression

At the current time, without the use of periodic DaT scanning, it is difficult if not impossible to determine from clinical signs and symptoms, alone, and in vivo whether or not a patient is suffering from a dopaminergic disorder, and if so, if the dopaminergic disorder is progressing. Thus, obtaining a baseline DaT level and pattern is useful, followed by periodic scans to determine if any changes can be discerned.

For example, in a patient whose baseline DaT level and pattern have been assessed by imaging and have been determined to be normal (baseline scan levels), it is possible to determine if a disorder subsequently manifests itself by detecting a change in the level and/or pattern of DaT binding. Thereafter, the time course of disorder progression can be monitored with subsequent imaging procedures at pre-determined periodic intervals. The subsequent scan results are compared to the baseline scan using both visual assessment and computerized analysis of the scanning results. A determination is then made by the physician regarding the progression of the disease as measured by DaT levels and patterns of imaging.

The present disclosure provides an in vivo method of optimizing a treatment regimen of a therapeutic formulation for a dopaminergic disorder in an organ of a mammalian subject. The method comprises administering a radiolabeled tropane to the subject treated with a first dose of the therapeutic formulation; measuring a level of tropane binding to the organ by acquiring a tomographic image; comparing the level of tropane binding to a projected level of tropane binding obtained from at least one tomographic image acquired before the administering step, and administering to the subject a second dose of the therapeutic formulation different than the first dose if the level of tropane is different than a projected level of tropane binding.

### 6. Monitoring of the Efficacy of Therapeutic Agent

Once baseline tropane binding levels and patterns have been assessed, and it has been determined that a patient is suffering from a dopaminergic disorder, the patient then undertakes a therapeutic regimen involving treatment with the drug of interest under the care of a physician or clinician skilled in treatment with such drugs. For example, the subject is treated with an initial dose of a first therapeutic drug, and the treated subject is then administered the radiolabeled tropane. The level and/or pattern of tropane binding to the organ is then measured. At pre-determined periodic intervals, the patient undergoes additional clinical evaluation and scanning to re-assess the DaT level and pattern. The subsequent scan results are compared to the baseline scan using both visual assessment and computerized analysis of the scanning results. A determination is made by the physician regarding the efficacy of the subject therapeutic in slowing or stopping the progression of the disease as measured by DaT levels. Depending on the new scanning and clinical evaluation data, the physician may adjust the dose of the subject drug or determine that it is in fact not effective.

The future effectiveness of a particular drug or dosage of that drug can be determined by comparing the level of tropane binding in the affected organ relative to a projected level of tropane binding, the projected level being obtained from the last one previously acquired by tomographic image. A difference in the level of tropane binding relative to the projected level of binding is indicative of the effectiveness of the therapeutic drug.

Longitudinally measuring presence of DaT as a means of assessing function of DaT assumes that Therapeutic interventions will have a feedback effect on activity and availability of DaT transporters (https://www.frontiersin.org/articles/10.3389/fnbeh.2014.00431/full), e.g., in the case of treatment with L-Dopa. However, due to feedback loops, drugs like L-Dopa can lead to further functional depression of DaT, which will result in DaT imaging that will show still decreasing DaT density and activity concomitantly with improved clinical symptoms. The feedback mechanism of L-Dopa put in place during treatment will then lead to an exacerbation of clinical symptoms when L-Dopa is withdrawn. Other therapeutics may have other mechanisms of action, impacting on the DaT in a different way. As a result, an increase or decrease in DaT binding by tropanes, is measured, depending on the underlying mechanism of action of the therapeutic that is being monitored.

When the concern is one particular dopaminergic condition, changes in the imaged patterns and levels of tropane binding can indicate whether a certain pharmacologic or non-pharmacologic treatment regimen is efficacious. For example, in the case of a neurodegenerative condition in the brain, a therapeutic effective drug converts an asymmetric period-shaped binding pattern seen in a PD brain into a symmetric comma-shaped binding pattern. But the drug may instead preserve the pattern seen in PD along different image acquisitions at different time periods, in effect indicating that PD is no longer getting worse, i.e., it is maintained at a certain binding level, and indicating that the density of DaT molecules is not further decreasing due to the action of the drug. When monitoring the time course of a dopaminergic condition and the efficacy of a therapeutic drug imaging provide information to ascertain whether the condition is improving, is being kept in check, or whether it is further deteriorating.

For example, in assaying for the efficacy of a drug for a dopaminergic condition affecting the brain, (e.g., Azilect® (rasagiline)), a labeled tropane such as [¹²³I]-2-carbomethoxy-3-(4-fluorophenyl)-N-(1-iodoprop-1-en-3-yl) nortropane is administered to the affected subject, and a SPECT tomograph of about 30 minutes in duration is acquired about 15 minutes after the administration. This serves as the first or initial tomograph, from which it can be determined an initial level of DaT binding (including a first density of DaT and a first amount of DaT) and pattern of DaT The term “level” encompasses the concepts of both the density and amount.

Subsequently, the drug is administered to the subject. After a period of time post-drug administration, the tropane-administration and tomograph-acquisition steps can be repeated to obtain a second tomograph, which reveals a second level of DaT Comparing the second level to the first level provides an indication as to if the drug is effective. For example, if the level has remained the same, this indicates that disorder at least has not progressed and that the drug is efficacious. Subsequently, additional cycles of tropane-administration and tomograph-acquisition steps are performed, from which obtain additional tomographs are obtained, and thus, additional DaT levels. Ultimately, a time course for the progression of the disease is constructed which is more objective than that obtained by subjective visual observations of a subject. Such a time course also enables comparisons of results across different research or clinical groups.

Creation of such time courses, which the disclosed methods enable, provides additional uses. For example, if time courses for a dopaminergic disorder progression are obtained from a number of individuals who have not been subjected to treatment, these time courses, or their various statistical averages, serve as useful controls for how progressive a disease is. In the case of a brain-related neurodegenerative disorder, a linear average value for the post-motor symptoms period slope of progression can be obtained, such as change in DaT levels divided by passed time. Alternatively, a hyperbolic, exponential, or multi-order polynomial model, can be fitted into the data to model it. Such models enable a prediction of how far the disease will have progressed at a certain time in the future; and can forecast a projected DaT level for a certain time in the future, enabling the testing of the effectiveness of a drug at that time point by comparing the DaT levels of a treated subject to those that are forecasted.

A time course for disease progression is obtained both to assess drug effectiveness, and to gain information about the natural disease progression in the absence of any treatment. When used during a treatment regimen, a time course can be used to optimize the treatment regimen. For example, by comparing the projected values from the time course to those found from the tomograph of the treated subject, a decision to: (1) change the drug; (2) change the dosage; or (3) add or remove a drug from the regimen can be made.

The disclosure also provides for an in vivo method of optimizing a treatment regimen of a therapeutic formulation for a dopaminergic disorder in an organ of a mammalian subject. In this method, a patient is administering a radiolabeled tropane after a first dose of the therapeutic drug or formulation. A measurement is then taken of the level of tropane binding to the organ by acquiring a tomographic image. A comparison is then made of the level of tropane binding to a projected level of tropane binding obtained from at least one tomographic image acquired before the administering step. A second dose of the therapeutic formulation is administered to the subject that is different than the first dose if the level of tropane is different than a projected level of tropane binding.

### 7. Dopaminergic Disorders

The provided methods can be used to assay for a variety of dopaminergic conditions, the time course of the disorder, and the effectiveness of a particular treatment. Among these are PD, ADHD, dementia, clinical depression, anxiety, narcolepsy, obesity, sexual dysfunction, schizophrenia, essential tremor, bipolar disorder, nausea/vomiting, addictive disorder (drugs, smoking), pheochromocytoma, or binge eating disorder. Other dopaminergic disorders include, but are not limited to, Hartnup disease, diabetes type I, polycystic ovary syndrome, clear cell renal carcinoma, and small cell lung cancer.

### 8. Pharmacological Agents

A variety of pharmacological agents which are neuroactive, which stop the progression of a dopaminergic disorder, or which may even reverse the detrimental effects of the disorder may be used to treat the disorder, and as such, can be assayed for efficacy according to the present methods.

As used herein, the term “neuroactive” encompasses compositions and drugs that are neuroprotective, disease-modifying, and/or symptom controlling with regard to neurological disorders. The term “neuroprotective” refers to that which serves to protect nerve cells against damage, degeneration, or impairment of function.

Neuroleptic drugs include, but are not limited to, chlorpromazine (Thorazine) (Generic Only); fluphenazine (Generic) Prolixin (Brand) (Novartis, East Hanover, N.J. or Bristol-Myers Squibb, New York, N.Y.); haloperidol (Generic) Haldol (Brand) (Ortho McNeill Janssen Pharmaceuticals, Raritan, N.J.); loxapine (Generic) Loxitane (Brand) (Actavis, Plc, Parsippany-Troy Hills, N.J.); perphenazine (Generic) Trilafon (Brand) (Schering-Plough, Kenilworth, N.J.); thioridazine (Generic) Mellaril (Brand) (Novartis Pharmaceuticals, East Hanover, N.J.); thiothixene (Generic) Navane (Brand) (Pfizer, Inc., New York, N.Y.); trifluoperazine (Stelazine) (Generic Only).

Newer drugs have been developed and launched which mitigate the negative side effects. Among these are: clozapine (Generic) Clozaril (Brand) (Novartis Pharmaceuticals, East Hanover, N.J.); aripiprazole (Generic) Abilify (Brand) (Otsuka Pharmaceutical Co. Ltd., Rockville, Md.); aripiprazole lauroxil (Generic) Aristada (Brand) (Alkermes Pharma, Athlone Co., Westmeath, Ireland); asenapine (Generic) Saphris (Brand) (Allergan, Plc, Westport Co., Mayo, Ireland); brexpiprazole (Generic) Rexulti (Brand) (Otsuka Pharmaceutical Co. Ltd., Rockville, Md.); cariprazine (Generic) Vraylar (Brand) Allergan, Plc, Westport Co. Mayo, Ireland); lurasidone (Generic) Latuda (Brand) (Sunovion, Marlborough, Mass.); paliperidone (Generic) Invega Sustenna/Invega Trinza (Brand) (Janssen Pharmaceutical, Raritan, N.J.); paliperidone palmitate (Generic) Invega Trinza (Brand) (Janssen Pharmaceutical, Raritan, N.J.); quetiapine (Generic) Seroquel (Brand) (Astra Zeneca, Cambridge, Engand); risperidone (Generic) Risperdal or Risperdal Consta (Brand) (Janssen Pharmaceutical, Raritan, N.J.); olanzapine (Generic) Zyprexa (Brand) (Eli Lilly and Co., Indianapolis, Ind.); ziprasidone (Generic) Geodon (Brand) (Pfizer, Inc. New York, N.Y.).

The dosages and particular neuroactive agents are known in the art (see, e.g., Allen (2013) Remington: The Science and Practice of Pharmacy (Pharmaceutical Press; London; 22nd ed.) as well as in Allen et al. (2001) Ansel's Pharmaceutical Dosage Forms and Drug Delivery Systems (Lippincott Williams & Wilkins; Philadelphia, 9th ed.)) are administered via a physician or clinician. The efficacy and effectiveness of each of these therapeutic agents is monitored. Non-limiting examples for neuroactive agents for PD include L-DOPA, bromocriptine, cabergoline, lisuride, pergolide, pramipexole, ropinirole, rotigotine, apomorphine, piribedil, rasagiline, and combinations thereof. Non-limiting exemplary neuroprotective agents for ADHD include amphetamine, dextroamphetamine, lisdexamfetamine, methamphetamine, methylphenidate, atomoxetine, clonidine, guanfacine, and combinations thereof. Non-limiting exemplary neuroactive agents for LBD include donepezil, rivastigmine, levodopa, melatonin, clonazepam, quetiapine, carbidopa-levodopa, and combinations thereof. Non-limiting exemplary neuroactive agents for clinical depression include fluoxetine, paroxetine, sertraline, citalopram, escitalopram, duloxetine, venlafaxine, desvenlafaxine, levomilnacipran, bupropion, trazodone, mirtazapine, vortioxetine, vilazodone, imipramine, nortriptyline, amitriptyline, doxepin, trimipramine, desipramine, protriptyline, tranylcypromine, phenelzine, isocarboxazid, selegiline, and combinations thereof. Therapeutic agents that are not yet known but are being or will be developed can be assessed as well.

Other pharmaceutical agents useful for treating other dopaminergic disorders which can be assayed by the methods of the disclosure include nicotinic acid and nicotinamide (for Hartnup (kidney) disease), insulin (diabetes type I), metaformin (diabetes 1), cancer (cytostatic drugs, antibody biologics, and radiation applied for the purpose of blunting tumor growth).

These therapeutic agents are administered to a subject at the various doses known by those physicians and clinicians who care for patients with the disorder. For example, a neuroactive agent is administered at about 0.05 mg/day to about 250 mg/day, or about 0.5 mg/day to about 25 mg/day, or about 1 mg/day to about 4 mg/day. The administration may be oral. Alternative routes of administrations are possible as well (e.g., transdermal, subcutaneous, intravenous).

Generally applicable methods of preparing medicinal or pharmaceutical formulations of neuroactive agents are well known in the art. In addition, various aspects of preparing medicinal or pharmaceutical formulations as well as including additional components (e.g., stabilizers, antibacterials, antifungals) in these formulations are described in Remington: The Science and Practice of Pharmacy, ibid. and in Ansel's Pharmaceutical Dosage Forms and Drug Delivery Systems, ibid. By using these resources in addition to relying on what is commonly known in the art, a person of skill in the art would be able to prepare the disclosed formulations, modify them to better suit individual situations, add additional components, and optimize concentrations of various components.

Reference will now be made to specific examples illustrating the disclosure. It is to be understood that the examples are provided to illustrate exemplary embodiments and that no limitation to the scope of the disclosure is intended thereby.

### EXAMPLES

### Example 1

### Assessment of Disease Modification with DaT2020

To determine if a therapeutic agent of interest is having the desired disease-slowing or disease-modifying effect on a patient, DaT imaging can be used. An initial (baseline) level and pattern of DaT is assessed. Then, determining if that level is normal or abnormal relative to the condition ids assessed. The therapeutic drug of interest is then administered, and the patient is then scanned again to determine if the DaT levels have changed (i.e., a follow-up scan). Both the baseline scan and the follow-up scan are accomplished following the steps below.

The patient is administered [¹²³I]DAT2020 under the direct supervision of a nuclear medicine physician or designee. For administration of [¹²³I]DAT2020, access into a large vein (e.g., antecubital vein) is established using a suitable indwelling polyurethane catheter that does not contain silicone (e.g., Bard® Poly Midline, C. R. Bard, Inc., Salt Lake City, Utah). To avoid extravasation of [¹²³I]DAT2020, correct localization of the catheter is ensured by a test injection of normal saline prior to the injection of [¹²³I]DAT2020.

The patient receives a single I.V. injection of [¹²³I]DAT2020 with a total activity dose amounting to 5.0 (±1.0 mCi.) The total administered radioactivity is of relevance and not the volume administered to achieve this dose. This single I.V. injection contains a maximum mass dose of DAT2020 of no more than about 16 ng and a total volume of up to about 5 mL. [¹²³I]DAT2020 must be administered manually via “slow I.V. injection”, followed by a 10 mL saline flush. As used herein “slow I.V. injection” refers to intravenous administration at about 5 ml/min to about 10 ml/min.

The exact radioactive dose administered is determined by calculating the difference between the radioactivity in the syringe and delivery system before and after injection. After the dose is delivered, the syringe is filled with a volume of saline equal to the administered dose volume. The syringe contents is recounted under the same conditions as used to determine the dose; separately. The delivery system is placed in a plastic container and counted in a dose calibrator (e.g., CRC®-25R Doe Calibrator, Capintec, Inc., Florham Park, N.J.) using the same parameters as used for the dose. Measured radioactivity values and times of measurement are documented in the source documents and recorded in the patient record, as well as the total injected volume. Injected radioactivity values outside the above stated range, i.e., values lower than about 4 mCi or higher than about 6 mCi are considered as potential sources of variation.

Specific SPECT scan parameters, including collimation and acquisition mode, are set out below.

Raw projection data is acquired into a 128×128 matrix, stepping each head 3 degrees for a total of 120 projections into a 20% symmetric photopeak window centered on 159 keV for a total scan duration of approximately 30 min.

Acquisition is in “step and shoot” mode with each head rotating 360 degrees using a parallel hole collimator supplied by the manufacturer of the SPECT gamma camera used to create the tomograph (GE Healthcare, Inc., Chicago, Ill.) to permit the possible reconstruction of a viable image (even if one head is faulty.)

The acquisition parameters are recorded for each subject at the time of the scan on the imaging source document.

The patient has voided and is otherwise comfortable and prepared to lie still for the length of time required for SPECT imaging using a SPECT camera with or without improved resolution capabilities (e.g., Discovery NM-630, GE Healthcare, Inc., Chicago, Ill. or inSPira HD®, Samsung Neurologica Corporation, Danvers, Mass.).

The subject is positioned in the camera and a peripheral 18 gauge to 22 gauge venous catheters inserted for the radiopharmaceutical infusion. A Y-system is used for optimal clearance of residual activity from the administration syringe.

After preparing the dose as per the above, the patient is positioned in the SPECT camera as described above. Subjects are injected with 5.0±1.0 mCi (296 MBq) [¹²³I]DAT2020. The subject is positioned in the camera at the time of injection, even though imaging will not commence until 15 min (±2 min) after radiotropane infusion.

[¹²³I]DAT2020 injection is administered by slow I.V. injection followed by a 10 mL saline flush. The start time of the injection is recorded along with the total volume injected.

The single SPECT acquisition is commenced within 15 min (±2 min) post-injection for a 30 min scan. Specifically bound is required to determine striatal activity which demonstrates peak uptake during this scan time window. Thus, ensure the accuracy of the acquisition start time once the subject is injected with [¹²³I]DAT2020.

The start and end times of the [¹²³I]DAT2020 scan are recorded on the imaging source document. Tomographs of the captured photon data are then compiled and read by a radiologist skilled in assessing DaT levels using imaging.

### Example 2

### Assessment of Disease Modification with DaTscan

The method described in EXAMPLE 1, above is used with the following changes.

The patient is administered [¹²³I]DaTscan under the direct supervision of a nuclear medicine physician or designee. The patient receives a single I.V. injection of [¹²³I]DaTscan with a total activity dose amounting to about 8.o mCi (±1.0 mCi.) The total administered radioactivity is of relevance and not the volume administered to achieve this dose. Injected radioactivity values outside the above stated range, i.e., values lower than about 7 mCi or higher than about 9 mCi are considered as potential sources of variation.

Subjects are injected with 8.0±1.0 mCi (296 MBq) [¹²³I]DaTscan. About 2.5 hrs to 3 hrs after radiotropane infusion the subject is positioned in the camera. The single SPECT acquisition is commenced for enough time to acquire at least 1.5×10⁶ counts (e.g., about 45 min) 30 min scan.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

What is claimed is:

1. A in vivo method for evaluating a therapeutic composition for its ability to modulate a dopaminergic activity in an organ of a mammalian subject having a dopaminergic disorder, comprising:
administering radiolabeled tropane to the subject;
determining a baseline level and pattern of binding of the administered radiolabeled tropane to dopamine transporters (DaT) in a the organ of the subject;
treating the subject with an initial dose of a first therapeutic composition;
administering a radiolabeled tropane to the treated subject; and
measuring the level and/or pattern of radiolabeled tropane binding to the organ of the treated subject,
a change in level and/or pattern of radiolabeled tropane binding in the treated subject relative to baseline levels and/or patterns of radiolabeled tropane binding being indicative of the ability of the therapeutic composition to modulate a dopaminergic activity in the organ of the subject.

2. The method of claim 1, wherein the change in the level of tropane binding is a decrease of at least about 5% to at least about 10%

3. The method of claim 1, wherein the change in the level of tropane binding is at least about 10%.

4. The method of claim 1, wherein the organ is the brain.

5. The method of claim 1, wherein the tropane is compound 2-carbomethoxy-3-(4-fluorophenyl)-N-(1-haloprop-1-en-3-yl) nortropane (DaT2020), 2-carbomethoxy-3-(4-fluorophenyl)-N-(1-iodoprop-1-en-3-yl) nortropane, or [I-123] N-ω-fluoropropyl-2β-carbomethoxy-3β-(4-iodophenyl) nortropane (DaTscan).

6. The method of claim 1, wherein the dopaminergic disorder is Parkinson's disease, attention deficit hyperactivity disorder, dementia, and clinical depression.

7. The method of claim 1, wherein tropane is radiolabeled with ¹²³I, ¹²⁴I, ¹²⁵I, ¹⁸F, ^{99m}Tc, ¹¹C, or ^{117m}Sn.

8. The method of claim 7, wherein the tropane is radiolabeled with ¹²³I, ¹²⁴I, ¹²⁵I, or ^{99m}Tc, or ^{117m}Sn, and the level and pattern of binding of radiolabeled tropane is measured by SPECT.

9. The method of claim 7, wherein the tropane is radiolabeled with ¹⁸F, ¹²⁴I, and ¹¹C, and the level and pattern of binding of radiolabeled tropane is measured by PET.

10. The method of claim 1, further comprising the steps of:
treating the subject with a secondary dose of the therapeutic composition which is different from the initial treatment dose if the level of radiolabeled tropane binding is decreased;
administering the radiolabeled tropane to the subject treated with the secondary dose; and
measuring the level of radiolabeled tropane binding,
an increase of at least about 10% in the level of radiolabeled tropane binding being indicative of the ability of the secondary dose of the therapeutic composition to positively modulate dopaminergic activity in the organ.

11. The method of claim 1, further comprising the steps of:
treating the subject with a second therapeutic composition which is different from the first therapeutic composition;
administering radiolabeled tropane to the subject treated with the second therapeutic composition; and
measuring the level and/or pattern of radiolabeled tropane binding,
a change of at least about 10% in the level and/or pattern of radiolabeled tropane binding being indicative of the ability of the first and second therapeutic compositions to modulate dopaminergic activity in the organ.

12. The method of claim 1, wherein the treating step comprises treating the subject with the first therapeutic composition and with a second therapeutic composition which is different from the first therapeutic composition;
a change in the level and/or pattern of radiolabeled tropane binding of at least about 10% being indicative of the ability of the first and second therapeutic compositions to modulate dopaminergic activity in the organ.

13. An in vivo method of obtaining a time course for the progression of a dopaminergic disorder in an organ of a mammalian subject, the method comprising:
carrying out a detection process, the detection process comprising;
administering a radiolabeled tropane to the subject; and
acquiring a first tomographic image of the organ; and
repeating the detection process to obtain a second tomographic image of the organ;
a difference in the second tomographic image relative to the first tomographic image being indicative of a change in the progression of the dopaminergic disorder in the organ of the subject.

14. An in vivo method of optimizing a treatment regimen of a therapeutic formulation for a dopaminergic disorder in an organ of a mammalian subject, the method comprising:
administering a radiolabeled tropane to the subject treated with a first dose of the therapeutic formulation;
measuring a level of tropane binding to the organ by acquiring a tomographic image; and
comparing the level of tropane binding to a projected level of tropane binding obtained from at least one tomographic image acquired before the administering step,
administering to the subject a second dose of the therapeutic formulation different than the first dose if the level of tropane is different than the projected level of tropane binding.

15. An in vivo method of determining effectiveness of a neuroprotective agent in a treatment of a mammalian subject having a dopaminergic disorder in an organ, the method comprising:
administering to the subject treated with the neuroprotective agent a radiolabeled tropane;
acquiring a tomographic image of the organ via SPECT or PET;
measuring a level of tropane binding in the organ from the tomographic image; and
determining the effectiveness of the neuroprotective agent based on the level of tropane binding in the organ relative to a projected level of tropane binding, the projected level of binding being obtained from at least one previously acquired tomographic image;
a difference in the level of tropane binding of at least about 10% being indicative of the effectiveness of the neuroprotective agent.

16. An in vivo method of detecting binding of a radiolabeled tropane to dopamine transporter (DaT) molecules in the brain of a mammalian subject, the method comprising:
administering the radiolabeled tropane to a subject;
initiating the acquisition of a tomographic image about 15 minutes after administering the tropane; and
terminating the tomographic image acquisition about 5 minutes to about 10 minutes after initiation,
a pattern of tropane binding to DaT molecules in the brain being obtained having two comma-shaped regions that are bilaterally symmetric with each if the brain of the subject is not affected by a dopaminergic disorder of the brain.
